(19) United States
(12) Patent Application Publication
(10) Pub. No.: US 2007/0054250 A1
(43) Pub. Date: Mar. 8, 2007

(54) Educational systems and methods for promoting writing skills

(76) Inventor: Cynthia Roth Pahr, San Diego, CA (US)

Correspondence Address: BUCHANAN, INGERSOLL & ROONEY LLP, P.O. BOX 1404, ALEXANDRIA, VA, 22313-1404, US

(21) Appl. No.: 11/514,754

(22) Filed: Sep. 1, 2006

Related U.S. Application Data
(60) Provisional application No. 60/713,954, filed on Sep. 2, 2005.

(51) Int. Cl. G09B 19/00; A61B 5/00

(52) U.S. Cl. 434/236; 600/300

(57) ABSTRACT

The disclosure relates to educational systems and methods of use thereof for improving student achievement, stimulating cognitive development, and increasing student motivation through improving writing skills. In accordance with the methods and devices of the disclosure students are able to build improved fine and gross motor skills.

CROSS REFERENCE TO RELATED APPLICATIONS

This application claims priority to U.S. Provisional Application Ser. No. 60/713,954, filed Sep. 2, 2006, the disclosure of which is incorporated herein by reference.

### TECHNICAL FIELD

The invention relates to educational systems and methods of use thereof for improving student achievement, stimulating cognitive development, and increasing student motivation through improving writing skills. In particular, the invention provides systems and methods to diagnose and improve fine and gross motor skills, visual, sensory, and organizational skills.

### COPYRIGHT NOTICE

Portions of the disclosure (e.g., paragraphs 4, 5, 19, 26, 38, 40, 42, 48-50, and 52-54) of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Subjects in classrooms throughout the country experience difficulty being attentive, have difficulty with writing, lose their place on a page and/or skip math problems Many subjects haven't developed the muscles needed for the fine and gross motor skills or have impaired sensory integration, which are important for a succesful classroom experience. Children often hide their difficulties by acting out, which sometimes leds to disciplinary problems. These behaviors are often a mask for hiding the true problem of being unable to complete class work well. Additionally, subjects who have sustained a brain injury often experience difficulty remembering the process and action of handwriting. These students (including both children and adults) need brain engaging strategies, direct teaching and guided practice to assist their progress toward improvement and successful completion of schoolwork, specifically written work.

There is less emphasis placed on direct teaching of fine and gross motor activities in classrooms even though students are expected to write neatly, keep their desks in order and follow directions-activities which involve developed motor skills. Additionally, many parents are unavailable to ensure daily play activities to develop these skills. While some subjects are able to achieve these goals with little direction, many have difficulty. Teachers and parents generally want to help subjects who have these difficulties, but without effective interventional strategies little progress is made.

Accordingly, there is a need to provide educational tools and methods for treating or diagnosing motor skill difficulties for use by professionals in the educational field, as well as parents and others. In addition, there is a need for methods and systems useful in building fine and gross motor skills, visual, sensory, and organizational skills or to compensate for skill difficulties, or merely to reinforce skills that students are already proficient in, which can be adapted for use in any curriculum or setting.

### SUMMARY OF THE INVENTION

The invention relates to a method for diagnosing or treating a motor skill disorder in a subject, comprising (a) preparing a surface with at least one set of at least three adjacent parallel bands having an essentially constant width, wherein each of the adjacent parallel bands has a different sensory form; (b) providing at least one exemplary letter or number that is properly placed on a surface within the three adjacent parallel bands; (c) allowing the subject to examine the exemplary letter or number and place one or more additional exemplary letters or numbers onto the surface; and (d) observing the ability of the subject to properly place the one or more additional exemplary letters or numbers on the surface, wherein proper placement is indicative of a subject with sufficient motor skill coordination.

The invention further relates to a kit, comprising (a) a surface with at least one set of at least three adjacent parallel bands having an essentially constant width, wherein each of the adjacent parallel bands has a different sensory form; and (b) a lettering or numbering means for locating a letter on the surface, wherein the lettering or numbering means is selected from a writing device, a tracing device, and a three-dimensional letter or number such that the three-dimensional letter or number is removably placeable on the surface within the three adjacent parallel bands.

The invention further relates to a system for encouraging proper writing comprising (a) a surface with at least one set of at least three adjacent parallel bands having an essentially constant width, wherein each of the adjacent parallel bands has a different sensory form; (b) at least one exemplary letter or number that is properly placed on a surface within the three adjacent parallel bands, wherein the at least one exemplary letter comprises arrows showing movement in a direction for forming the at least one exemplary letter; and (c) at least one writing device, tracing device, or three-dimensional letter or number.

The invention further relates to a method for developing motor skills, comprising contacting a thin support with a magnet from the underside of the surface, the surface being substantially planar and sufficiently thin to allow magnetic radiation to pass there-through and having on its surface at least three parallel bands, each band comprising a different sensory form, and an exemplary design, the design comprising arrows depicting a direction of movement; placing a magnetic metal on the top of the surface; and moving the magnetic metal on the surface of the thin support using the magnet located on the underside, wherein a subject traces the exemplary design and wherein the subject't handj is substantially lhidden from view by the thin support.

The invention further relates to a writing system, comprising a stylus; a malleable substance; and a design, wherein a subject contacts the malleable substance with the stylus such that force is needed to deform the malleable substance with the stylus and the subject traces the design on the malleable substance.

Other aspects of the invention are described throughout the specification and the claims.

### BRIEF DESCRIPTION OF THE DRAWINGS

FIG. 1 depicts a representative surface containing sets of three adjacent parallel bands of equal width, separated from one another by open spaces.

FIG. 2 depicts a representative letter “g” placed within a set of parallel bands, as well as the starting point (closed circle) and direction (dotted lines and arrows) in which the “g” would be formed on the surface within the set of bands (i.e. the “indicia”).

FIGS. 3A-C depict a surface with exemplary letters placed on the surface within the three parallel bands, with arrows depicting a direction of movement. FIG. 3C depicts a magnetic tracer used to trace the letters.

FIG. 4 depicts a subject crossing the midline of his body.

FIG. 5 shows exemplary letters and numbers including arrows depicting the direction for tracing or copying the letters and numbers.

### DETAILED DESCRIPTION

The invention relates to educational systems and methods of use thereof for improving student achievement, stimulating cognitive development, and increasing student motivation through improving writing skills. In particular, the invention provides systems and methods to diagnose and improve fine, gross motor skills, visual, sensory, and organizational skills.

The invention assists subjects with the development of motor skills necessary to write successfully. In addition to assisting subjects with motor or visual difficulties, the systems of the invention can be used to reinforce the confidence of students that do not outwardly express any difficulty with motor or visual skills, but may just need practice to write neater and more legibly. Integrating motor activities into the classroom and home environment can be a tremendous help in reading, writing, attention, and memory. Subjects who learn to develop specific motor movements can incorporate such skills into successful participation in school activities. Early sensorimotor learning experiences are fundamental building blocks for later, more complex perceptual and cognitive development. Memory is also improved by using motor movements, auditory and visual stimulation during the learning process. Subjects with learning disabilities, Attention Deficit Hyperactivity Disorder, Mental Retardation, Autism and Tourette Syndrome particularly benefit from this multisensory approach to learning and writing. Accordingly, in one aspect, the invention provides means for identifying the population of subjects that would most benefit from the systems of the invention.

In one embodiment, the system of the invention comprises a method for diagnosing or treating a motor skill disorder in a subject, comprising (a) preparing a surface with at least one set of at least three adjacent parallel bands having an essentially constant width, wherein each of the adjacent parallel bands has a different sensory form; (b) providing at least one exemplary letter or number that is properly placed on a surface within the three adjacent parallel bands; (c) allowing the subject to examine the exemplary letter or number and place one or more additional exemplary letters or numbers onto the surface; and (d) observing the ability of the subject to properly place the one or more additional exemplary letters or numbers on the surface, wherein proper placement is indicative of a subject with sufficient motor skill coordination.

The surface is essentially any flat surface and can be formed from any substrate, for example: a textile product, such as fabric; a paper product, such as writing paper of any thickness; a heavier weight substrate such as cardboard or poster board. In addition, the surface may be a typical schoolroom blackboard or whiteboard; a computer or other electronic device display screen; or any other accessible surface, such as a wall, a desktop, a door, a laminate and the like.

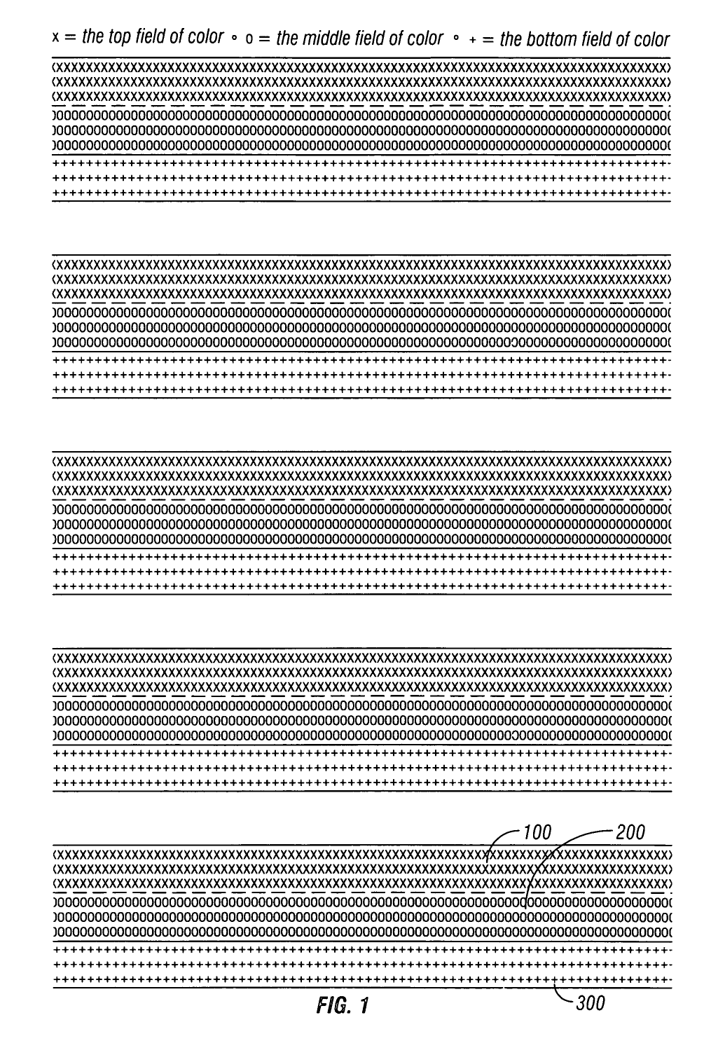

The at least three adjacent parallel bands across the surface, each with an essentially constant width refers to the grouping of bands together in at least “threes,” usually separated by an open space of any width, as shown in FIG. 1. It is also intended that the parallel bands corresponds to a “row” upon which letters and numbers are placed on the surface, such as a row of text in a book.

The bands can comprise different sensory form sufficient to distinguish one band from the other. For example, the bands can comprise tactile differences (rough, smooth, etc.). In another embodiment, the “different sensory form” can comprise different colors, shading, fill patterns, or a combination of all of the above differences, so long as the user can distinguish the difference between the bands. The exemplary letter may be permanently or removably positioned on the surface. Exemplary letters may comprise a fixed font form on a surface, a foam material, a wood material, a plastic material, a paper material or combinations thereof.

Writing devices are known in the art. For example, a writing device may be a pen, pencil, crayon, marker and the like.

In a particularly useful embodiment, the three parallel bands are depicted in FIG. 1 in three different sensory forms as “X” (100), “O” (200) and “+” (300), from top to bottom, as follows:
X—to represent sky (100)
O—to represent grass (200)
+—to represent earth (300)
   It will be recognized that the sensory forms can be any color, texture, hatching and the like, so long as the user can differentiate the locations on the surface. In one aspect, band 100 will be blue (“sky”), band 200 will be green (“grass”) and band 300 will be brown (“earth”).

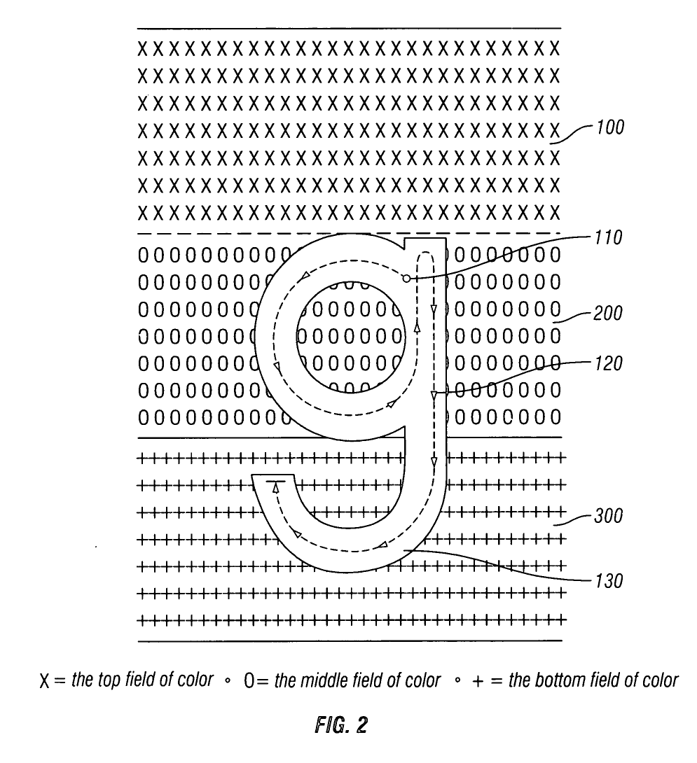

The reason why this is such a meaningful pattern is that children can relate to sky on top of grass, and grass on top of earth. In this embodiment (as well as others), letters can be grouped in terms of which of the three bands their shapes extend into. For example, students can be shown which letters “live” in the grass only, sky and grass, and in the grass and earth, such as:
Grass letters—a,c,e,i,m,n,o,r,s,u,v,w,x,z—because they all fit and are written in the middle of the three adjacent parallel bands.
Sky and Grass letters—b,d,f,h,k,l,t—The “ascenders” rise above the middle bands into the top band.
Grass and Earth letters—g, p,q,y—“descenders” extend below the middle band (see, e.g., FIG. 2 portion 130).
Sky, Grass and Earth letter—j—the letter extends to lower band, while the “dot” is in the upper band.

By reinforcing these categories of letters, the students are even better prepared to properly position them on the surface.

Tracing devices are known in the art. For example, a tracing device may be a magnet and a counter magnetic metal, a stylus and the like.

One aspect to the practice of the invention is to provide the student with, not only a blank (or partially blank) surface, but also with at least one letter or number that is placed on a “surface mimic” (i.e. on a background that has the same pattern of adjacent parallel bands with different sensory forms) as shown in FIG. 2, so that the student can see from looking at the letter on the surface mimic where it should be positioned on the blank surface within the three adjacent parallel bands.

In addition, the exemplary letter or number may include, within or in the proximity of, a “guide” in the form of a starting point and direction in which the letter or number is best drawn on the surface. Referring to FIG. 2, the formation of the letter “g” is depicted in solid black, with the starting point for formation of the letter expressed as a closed white circle 110, and a dashed line with arrows 120 indicating the direction in which the letter is formed (i.e. letter formation indicia). Also depicted are the different sensory forms 100, 200, and 300 forming substantially parallel bands. Either together or separately, the student can practice forming the letter “g” using the exemplary letter or number, and then practice placing it in the right location on a surface.

The exemplary letter or number may be placed on its own separate surface, including a card, plastic, or other such surface, on which a student can trace the letter as indicated by the optional indicia. The exemplary letter may also be shaped in the form of a letter, with the portions of the letter corresponding to the bands on which the letter needs to be placed on the surface.

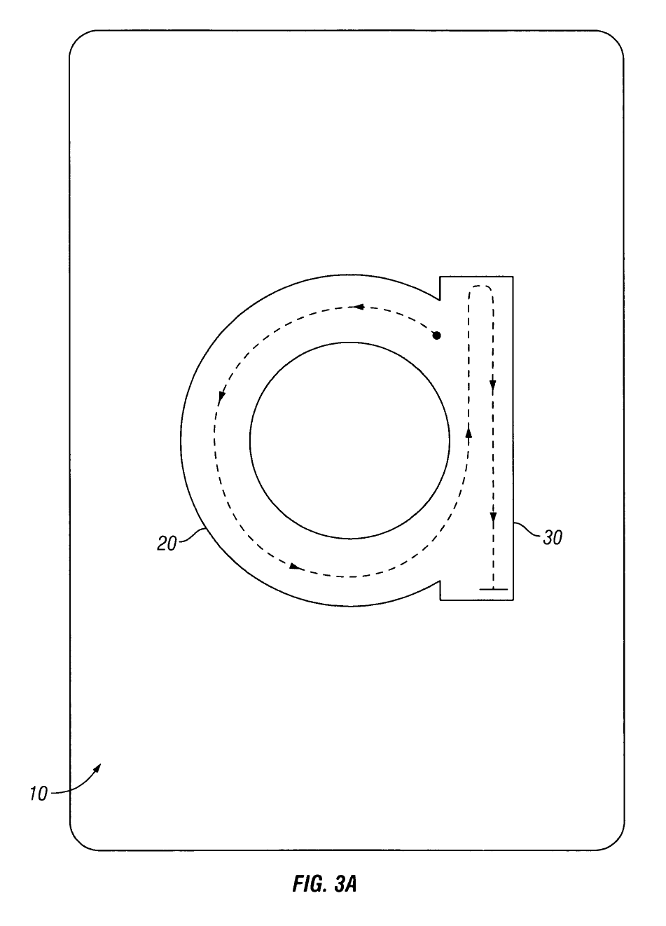

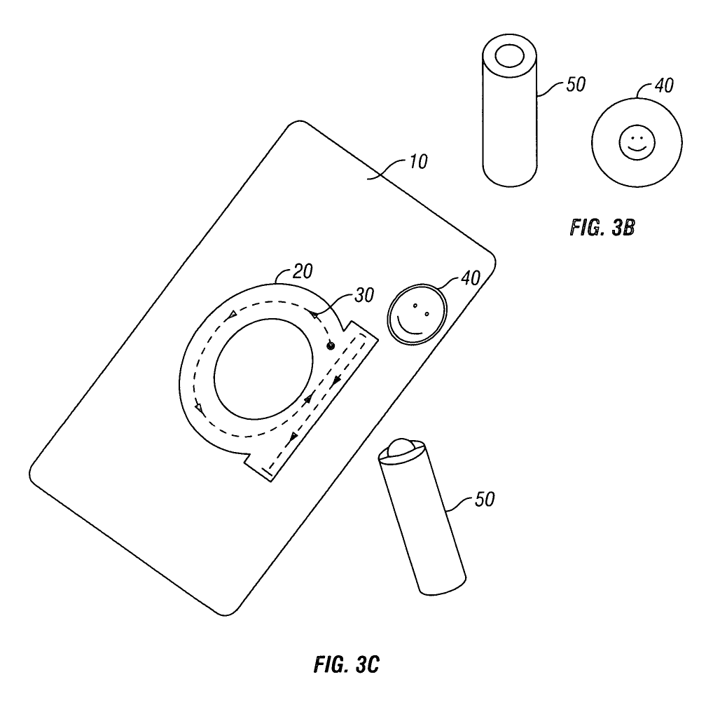

In a particularly useful embodiment, the tracing device comprises directions on how to trace the design, wherein arrows are depicted on the exemplary letter. See for example FIG. 3A-C. In FIG. 3A, the surface (10) is depicted with a design (20), and arrows (30) showing directional instructions on how to trace the design. FIG. 3C further depicts the magnet (40) and magnetic tracer (50) for use by a subject to trace exemplary designs on the surface.

Another aspect of the invention relates to a kit, comprising (a) a surface with at least one set of at least three adjacent parallel bands having an essentially constant width, wherein each of the adjacent parallel bands has a different sensory form; and (b) a lettering or numbering means for locating a letter on the surface, wherein the lettering or numbering means is selected from a writing device, a tracing device, and a three-dimensional letter or number such that the three-dimensional letter or number is removably placeable on the surface within the three adjacent parallel bands.

The invention further relates to a system for encouraging proper writing comprising (a) a surface with at least one set of at least three adjacent parallel bands having an essentially constant width, wherein each of the adjacent parallel bands has a different sensory form; (b) at least one exemplary letter or number that is properly placed on a surface within the three adjacent parallel bands, wherein the at least one exemplary letter comprises arrows showing movement in a direction for forming the at least one exemplary letter; and (c) at least one writing device, tracing device, or three-dimensional letter or number.

The invention further relates to a method for developing motor skills, comprising contacting a thin support with a magnet from the underside of the surface, the surface being substantially planar and sufficiently thin to allow magnetic radiation to pass there-through and having on it's surface at least three parallel bands, each band comprising a different sensory form, and an exemplary design, the design comprising arrows depicting a direction of movement; placing a magnetic metal on the top of the surface; and moving the magnetic metal on the surface of the thin support using the magnet located on the underside, wherein a subject traces the exemplary design and wherein the subject's hand is substantially hidden from view by the thin support.

The invention further relates to a writing system, comprising a stylus; a malleable substance; and a design, wherein a subject contacts the malleable substance with the stylus such that force is needed to deform the malleable substance with the stylus and the subject traces the design on the malleable substance. The stylus has the appearance of a pen, and a substantially pointed tip, and the malleable substance is clay, or the like.

In its simplest form, the system of the invention comprises a surface with sets of three adjacent parallel bands as shown in FIG. 1 (100, 200 and 300), separated by open spaces, onto which sets of exemplary letters and/or numbers are printed in their proper location within the three adjacent parallel bands, but having enough remaining space on the parallel bands for a student to practice copying the printed letters onto the blank spaces in their proper location. In other words, the “surface mimic” with the letters/numbers may simply be at a different location on the same surface as the surface on which the letters are placed (either adhesively or drawn) during the practice of the invention.

Teachers and others can help subjects improve motor skills using simple exercises and interventions as described herein within or outside the classroom setting. Specific motor methods and systems are provided to help subjects improve their skills that are easily performed on a regular basis obtain the best results. The invention assists subjects having various perception and motor skill disorders to develop skills that enable them to properly function in school, work, etc.

For example, many times, frustration is a result of the inability to complete a task in a timely fashion or with little success. It is very important to foster continued development of motor skills while being sensitive to the limitations of the subject.

One developmental activity is hand dominance. Typically, children develop hand dominance by the age of six. If there is frequent switching between hands for a given task and neither hand is doing a good job, then there is a problem.

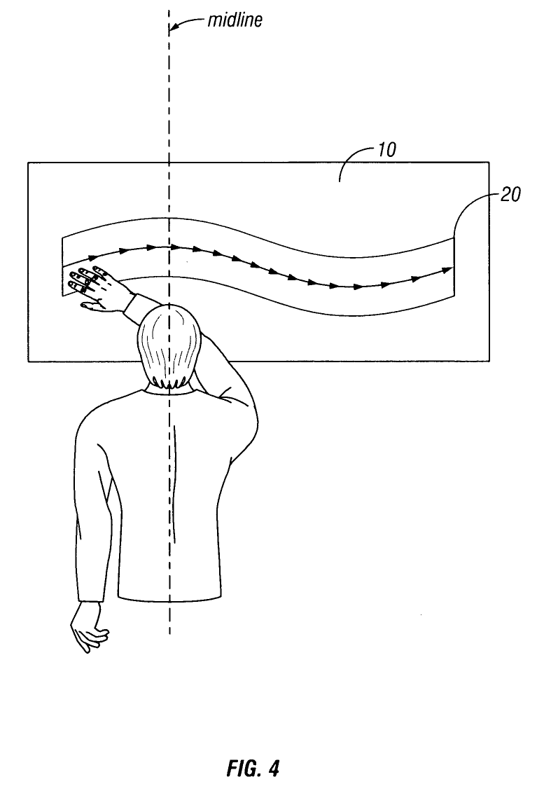

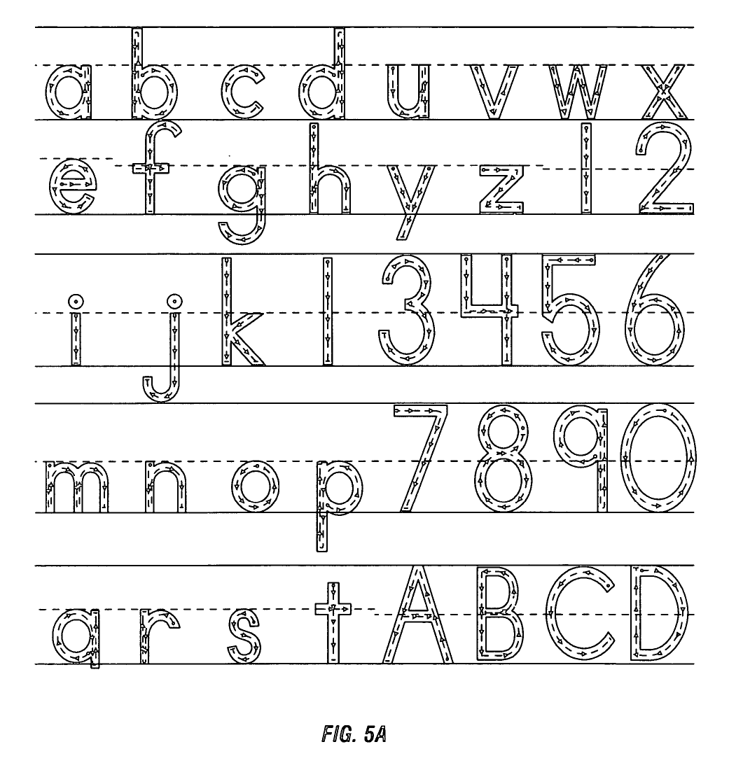

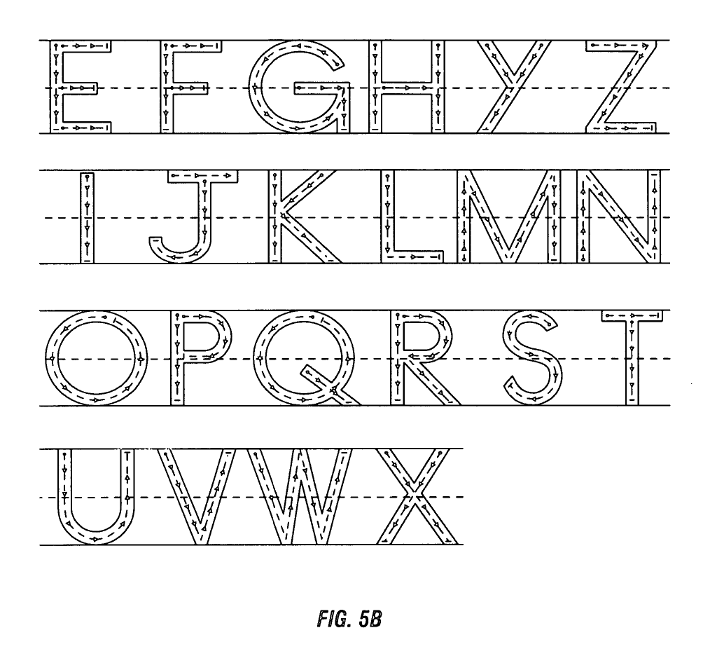

Subjects who have undeveloped hand dominance may be having difficulty learning or writing due to the inability to cross their hands over their midline (the vertical center of the body). Consequently, when their hand reaches the center, they transfer their writing utensil to the other hand in order to complete a task. Crossing midline is a skill which engages both sides of the brain. This helps coordination of both eyes, ears, hands and feet. Developing this skill can lead to increased learning and success, for example, in school activities. Using the invention, subjects will engage the use of the presumed dominant hand while also engaging the presumed non-dominant hand. The invention is designed to be a fun tool, which then helps keep a child's mind off of the difficulty with dominance while also strengthening the dominant hand. Thus, in one aspect of the invention, a subject traces a design by placing a magnetic metal on the top of a surface and moves the magnetic metal using the magnet located on the underside to trace an exemplary design. As depicted in FIG. 4 a surface (10) with a design (20) is depicted. The midline of the subject is identified. During use the subject traces the design (20) such that his writing arm crosses his midline. Design (20) may be any letter, number, word, word phonic or design. The surface and design should be sufficiently larger to enable the subject to cross the midline.

Difficulties with letter, number and shape formation often are directly related to the integration of a subject's motor ability. Practicing gross motor movements and activities can help a subject integrate both sides of the body. Those who have difficulty crossing midline, with directionality or with reversals often have difficulty with producing forms. Subjects with motor planning or perceptual problems need visual cues to assist them until their bodies develop motor memory of how to make the various forms. Visual difficulties can also be a factor. Using the invention will engage the use of both sides of the body while completing a fun learning activity.

Sensory integration refers to the neural organization of sensory information for functional behavior. Sensory integration problems are a condition of the inability to effectively process sensory information to affect an appropriate motor action, and a symptom of many diseases. For example, sensory integration problems may be a symptom of sensory integration dysfunction, Attention Deficit Hyperactivity Disorder, Autism, Mental Retardation or Tourette Syndrome. Sensory integration problems are generally found in children, although it may also be found in adults with disabilities or brain injuries. Many problems have been associated with the disorder, including impaired motor function. The major effect of sensory processing disorder is the impairment of either fine or gross motor skills, resulting from impaired sensory messages to the brain. In most instances, handwriting is affected.

Subjects, including children, with sensory integration problems find it difficult to move purposefully and have a hard time resisting the pull of gravity. A main concern of occupational therapists and parents is that a child's fine motor skills may be affected due to the sensory integration problems. If this is the case, the child may experience difficulty using a writing instrument such as a pen or pencil to write. Because of the difficulty of controlling a writing instrument for a child having impaired fine motor skills, it is often difficult to decipher the child's handwriting even if the child is able to write.

Various treatments and therapies have been developed to combat the problems created by sensory integration problems. Early intervention and therapy is important in children displaying symptoms of the disorder. In therapy, depending on the individual case of sensory integration problems, a child may go through programs including early education, and speech, occupational and/or physical therapy. For children whose fine motor skills are affected by sensory processing disorder, there are few options available.

Poor tactile perception is a common disorder of subjects with sensory integration problems. Subjects who have difficulty distinguishing tactile articles or mediums accurately have poor tactile perception. Typically, fine motor skills are affected when a subject has poor tactile perception. Poor tactile perception impedes the sensory feedback that is needed to correctly guide motor tasks, for example, to write with a pencil, cut with scissors, or hold a piece of paper.

A writing system of the invention is useful in developing and promoting fine motor skills and muscle strength. Accordingly, in another aspect of the invention a writing system is provided comprising a stylus and malleable substance comprising a malleable substance (e.g., clay). This writing instrument assist persons suffering from sensory integration problems to enable such persons to more effectively write by forcing the subject to press down sufficiently enough on the stylus to provide stronger sensory feedback and deform the malleable substance. In one aspect of the invention, the stylus is made of wood, metal and/or plastic. The stylus comprises a first end and a second end, wherein at least one end is tapered to provide a point. The point of the stylus need not be sharp, but will typically be sufficiently pointed that it resembles a pen or pencil. The malleable substance can comprise clay (e.g., PLAY-DOH™) or other material that is deformable and retains deformation upon use for a sufficient period of time. In one aspect of the invention, the writing system comprises a stylus and clay and optionally includes one or more design or letter templates. During use, the clay is deformed when the subject applies sufficient pressure to the writing instrument.

Subjects who have trouble keeping their place on a page may need exercises to help train their eyes to work more efficiently. Tracking problems can be helped by integrating some eye exercises which can be done in large groups. Having the ability to track efficiently requires the eyes to move in unison and follow moving objects or words on a page with a smooth motion. The invention provides a medium which helps train the eye to track an object directed in a familiar motion. Thus, in the methods and systems of the invention, a surface comprising at least 3 bands of different sensory forms (e.g., color, gray scale, markings) are used to assist the user to visually discriminate and track location on the surface during use.

The ability to identify an object and match it to an identical object is called visual discrimination. Visual perception is involved in identification of objects based on the ability to interpret what one sees based on past experience. Subjects who have difficulty with letter, number or shape discrimination need to develop awareness of the forms in a variety of ways. The invention provides a brain engaging format to assist in improving letter discrimination.

Subjects may be disorganized due to poor perceptual-motor coordination, poor visual-motor skills, attention problems or difficulty in following direction. Improvement in this area may be made by breaking the task into smaller pieces, verifying that the subject understood the instructions, “motoring through” (physically moving the subject's hand) and verbalizing the action simultaneously, giving greater sensory information.

For subjects with motor problems, just modeling the action desired is not enough instruction. Tracing helps build confidence and success in writing activities. Once a subject feels more confident, then the subject is open to learning proper formation of letters.

Some subjects have great difficulty guiding their motor movements without looking. The motor tasks required in school are slowed down by their necessity to watch their own movements in attempting to make them correctly. For subjects with difficulty moving in a sensory way to complete a task, the invention forces him to feel the motion of his hand while not being able to actually see his hand move. The subject sees the result of his movement in the magnet.

Spatial awareness is the perception of one's body and objects around them and relies on a good perceptual-motor foundation. Spatial awareness relates not only to how one controls his own body movements but on how he perceives pictures, letters and numbers and those pictures, letters and numbers around them.

Imitating movements seems like a simple task but to a subject with sensory problems it may be a source of frustration and failure. For example, many times throughout a school day students are asked to imitate the teacher's direction. It is often not realized how often it involves copying a physical movement. If students are having trouble imitating movements or following physical directions, they may need exercises to increase sensory information to the brain, and will undoubtedly exhibit difficulty with motor skills. The invention provides opportunities for the subject to practice and develop the ability to properly imitate movements with respect to letter, number and shape formation.

The system of the invention is designed to assist subjects with motor and/or visual processing difficulties (as well as new learners) to develop the skills necessary to write better and, more particularly, to place letters and words in their proper position on a surface, such as a piece of paper.

Visual and auditory cues access other modalities which the student can use to assist in writing letters in the proper place. As discussed above, poor eye-hand coordination may also be a factor that may influence writing skills that would be improved using the inventive system.

Articles of manufacture are also contemplated by the invention. Such articles of manufacture will comprise one or more components (e.g., writing system components) and instructions explaining the use of the components to improve writing skills and/or fine motor skills.

The information presented above is provided to give those of ordinary skill in the art with a complete disclosure and description of how to make and use the preferred embodiments of the invention, and is not intended to limit the scope of what the inventor regards as the invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method for diagnosing or treating a motor skill disorder in a subject, comprising:
(a) preparing a surface with at least one set of at least three adjacent parallel bands having an essentially constant width, wherein each of the adjacent parallel bands has a different sensory form;
(b) providing at least one exemplary letter or number that is properly placed on a surface within the three adjacent parallel bands;
(c) allowing the subject to examine the exemplary letter or number and place one or more additional exemplary letters or numbers onto the surface; and
(d) observing the ability of the subject to properly place the one or more additional exemplary letters or numbers on the surface, wherein proper placement is indicative of a subject with sufficient motor skill coordination.

2. The method of claim 1, wherein the surface is selected from the group comprising of a chalk board, a paper, a dry-erase board, a computer and a laminate.

3. The method of claim 1, wherein the at least one exemplary letter is positioned in the middle or middle band, the middle and top band, or the middle and lower band of the three adjacent parallel bands.

4. The method of claim 3, wherein the at least one exemplary letter is permanently positioned on the surface.

5. The method of claim 3, wherein the at least one exemplary letter is removably positioned on the surface.

6. The method of claim 5, wherein the removable at least one exemplary letter comprises a foam material, a wood material, a plastic material, a paper material or combinations thereof.

7. The method of claim 1, wherein the different sensory form comprises different colors.

8. The method of claim 1, wherein the different sensory form comprises different shades.

9. The method of claim 1, wherein the different sensory form comprises different markings.

10. The method of claim 7, wherein the parallel bands comprise blue, green, and brown.

11. The method of claim 10, wherein the top band of the at least three parallel bands is blue, the middle band is green, and the bottom band is brown.

12. The method of claim 1, wherein the subject placed one or more additional exemplary letters or numbers on the surface using a writing or tracing device.

13. The method of claim 12, wherein the writing device is selected from the group comprising of a pen, pencil, crayon, marker and the like.

14. The method of claim 12, wherein the tracing device comprises a magnet.

15. The method of claim 14, wherein the tracing device comprises a magnet and a counter magnetic metal.

16. The method of claim 1, wherein the exemplary letter further comprises directions on how to trace the letter comprising arrows depicted on the exemplary letter.

17. A kit, comprising:
(a) a surface with at least one set of at least three adjacent parallel bands having an essentially constant width, wherein each of the adjacent parallel bands has a different sensory form; and
(b) a lettering or numbering means for locating a letter on the surface, wherein the lettering or numbering means is selected from a writing device, a tracing device, and a three-dimensional letter or number such that the three-dimensional letter or number is removably placeable on the surface within the three adjacent parallel bands.

18. A system for encouraging proper writing comprising:
(a) a surface with at least one set of at least three adjacent parallel bands having an essentially constant width, wherein each of the adjacent parallel bands has a different sensory form;
(b) at least one exemplary letter or number that is properly placed on a surface within the three adjacent parallel bands, wherein the at least one exemplary letter comprises arrows showing movement in a direction for forming the at least one exemplary letter; and
(c) at least one writing device, tracing device, or three-dimensional letter or number.

19. The system of claim 18, wherein the surface is selected from the group comprising of a chalk board, a paper, a dry-erase board, and a laminate.

20. The system of claim 18, wherein the at least one exemplary letter is positioned in the middle band, the middle and top band, or the middle and lower band of the three adjacent parallel bands.

21. The system of claim 20, wherein the at least one exemplary letter is permanently positioned on the surface.

22. The system of claim 20, wherein the at least one exemplary letter is removably positioned on the surface.

23. The system of claim 22, wherein the removable at least one exemplary letter comprises a foam material, a wood material, a plastic material, a paper material or combinations thereof.

24. The system of claim 18, wherein the different sensory form comprises different colors.

25. The system of claim 18, wherein the different sensory form comprises different shades.

26. The system of claim 18, wherein the different sensory form comprises different markings.

27. The system of claim 24, wherein the parallel bands comprise blue, green, and brown.

28. The method of claim 27, wherein the top band of the at least three parallel bands is blue, the middle band is green, and the bottom band is brown.

29. The method of claim 28, wherein the writing device is selected from the group comprising of a pen, pencil, crayon, marker and the like.

30. The method of claim 18, wherein the tracing device comprises a magnet.

31. The method of claim 30, wherein the tracing device comprises a magnet and a counter magnetic metal.

32. A method for developing motor skills, comprising:
contacting a thin support with a magnet from the underside of the surface, the surface being substantially planar and sufficiently thin to allow magnetic radiation to pass there-through and having on it's surface at least three parallel bands, each band comprising a different sensory form, and an exemplary design, the design comprising arrows depicting a direction of movement,
placing a magnetic metal on the top of the surface; and
moving the magnetic metal on the surface of the thin support using the magnet located on the underside, wherein a subject traces the exemplary design and wherein the subject's hand is substantially hidden from view by the thin support.

33. A writing system, comprising:
a stylus;
a malleable substance; and
a design wherein a subject contacts the malleable substance with the stylus such that force is needed to deform the malleable substance with the stylus and the subject traces the design on the malleable substance.

34. The system of claim 33, wherein the stylus has the appearance of a pen.

35. The system of claim 34, wherein the stylus has a substantially pointed tip.

36. The system of claim 33, wherein the malleable substance is clay.
